(19) United States
(12) Patent Application Publication
(10) Pub. No.: US 2007/0276434 A1
(43) Pub. Date: Nov. 29, 2007

(54) VASCULAR SHEATH

(75) Inventor: John C. Opie, Scottsdale, AZ (US)

Correspondence Address: SNELL & WILMER L.L.P. (Main), 400 EAST VAN BUREN, ONE ARIZONA CENTER, PHOENIX, AZ, 85004-2202, US

(73) Assignee: JS Vascular, Inc., Scottsdale, AZ (US)

(21) Appl. No.: 11/567,851

(22) Filed: Dec. 7, 2006

Related U.S. Application Data
(63) Continuation of application No. 11/042,472, filed on Jan. 24, 2005, now abandoned.
(60) Provisional application No. 60/538,712, filed on Jan. 23, 2004.

(51) Int. Cl. A61B 17/12

(52) U.S. Cl. 606/213

(57) ABSTRACT

Disclosed is a vascular sheath for helping to prevent bleeding during procedures in which devices must be inserted into a blood vessel such as an artery or vein. The vascular sheath includes at least one manually compressible primary seal that has a lumen passing therethrough. A device inserted into the blood vessel first passes through the sheath, and thus through the lumen in the primary seal. By manually adjusting the compression of the primary seal the size of at least part of the lumen is made to substantially conform to the outer surface of the device. The primary seal can thus seal against large and small sized devices to prevent bleeding. It is preferred that a secondary seal also be used and one type of secondary seal is a flexible disk or flap with one or more slits through which the device passes.

### CROSS-REFERENCE TO RELATED APPLICATIONS

This application is a continuation of and claims priority to U.S. patent application Ser. No. 11/042,472 also entitled “VASCULAR SHEATH”, filed on Jan. 24, 2005, which application claims priority to U.S. Provisional Application Ser. No. 60/538,712, filed Jan. 23, 2004, by inventor John C. Opie, both of which are incorporated herein by reference.

### FIELD OF THE INVENTION

The present invention relates to medical systems and methods, and more particularly, to a vascular sheath to assist in preventing excessive bleeding during certain medical procedures.

### BACKGROUND OF THE INVENTION

This invention relates to vascular sheaths (preferably larger diameter sheaths) having an improved hemostatic valve or gasket assembly to assist in preventing excessive bleeding when the sheath is “dormant.” “Dormant” in this context means that the sheath is temporarily transmitting and/or retaining a small diameter secondary device such as a medical guide wire (also referred to herein as a guide wire or wire), or diagnostic catheters for procedures such as serial angiograms.

Vascular sheaths (also referred to herein as a sheath or vascular access sheath) are delivery platforms used to introduce secondary devices into blood vessels. These secondary devices include, for example, dilators, guide wires, angioplasty balloons, stents, atherectomy catheters, angiography catheters and abdominal aortic aneurysm endo-luminal grafts. The sheaths usually range from a diameter of about 5-French to 24-French (“Fr”) depending upon the size of the secondary device. The upper limit is dictated to some extent by human anatomy, particularly the size of the femoral artery.

Known sheaths work relatively well and are substantially hemostatic when used with relatively large indwelling secondary devices. However, when known sheaths are used with a relatively small diameter secondary device, such as a guide wire, they typically leak sizable quantities of blood. This is due to the efficiency of the cruciate slits typically found in the elastomeric (usually a silicone rubber) gasket that is used in known sheaths to form a seal. Using the example of a guide wire, the wire tends to slip into one of the slits creating a small eye-shaped opening in the slit and bleeding occurs through the opening. Because of this, it is common to put a second sheath, usually of 10-French diameter, over the wire and into the larger sheath to create a seal and stop the bleeding. In some instances a glob of wax is used to plug the end of the sheath.

One solution to this problem has been suggested by the Touhey-Borst system, which is known in the art. However, that system does not perform well when large bore secondary devices (such as large bore obdurators) are removed from large bore sheaths and only wires or catheters remain. The Touhey-Borst valve construction includes an O-ring seal that is compressed during use. However, the O-ring is contained statically within the distal end of a second chamber. Such a mechanism is unable to seal a large bore secondary device, and after the large device is removed, then seal down against a small diameter secondary device, such as a wire or angiocatheter. This is due to the fact that only so much compression is available with the non-moving O-ring.

Other methods have been developed to solve this problem and have not been entirely successful. Some sheaths include two or even three elastomeric gaskets, but blood still leaks when only the wire passes through the sheath. Other sheaths include torroidal balloons. Torroidal balloons may work but are cumbersome and when a large secondary device is removed from the sheath one must quickly inflate the balloon with a syringe to avoid a sudden and large blood loss via the large opening through the balloon.

Other devices have suggested iris-type valve assemblies, but these have not been widely used due to the expense of making them and the potential problem of engaging them or disengaging them with resultant transient torrential femoral artery bleeding. Still other inventors have devised flapper valve mechanisms.

### SUMMARY OF THE INVENTION

The invention is a vascular sheath that permits the passage of a secondary device into a blood vessel, such as the femoral artery. In accordance with the present invention, an improved vascular access sheath is provided to facilitate the introduction of both large and small diameter secondary devices into a vein or artery, while assisting to prevent significant blood loss, even when the sheath only transmits a relatively small secondary device, such as a medical guide wire.

The sheath includes a body and a primary seal retained in the housing. The primary seal has a lumen passing therethrough and the secondary device passes through the lumen. As the primary seal is compressed (which is preferably done by tightening a cap on the body, wherein the cap is attached to a post that presses against the primary seal) at least part of the lumen is compressed and substantially presses against the outer surface of the secondary device to form a seal. In this manner, the sheath can seal against both relatively large diameter devices and relatively small diameter devices.

The sheath also preferably includes one or more secondary seals. The preferred secondary seal is a flexible disk having one or more slits through which the secondary device can pass.

A vascular access sheath according to the invention is preferably is a large bore vascular access sheath of a size between 5 Fr and 24 Fr.

It is to be understood that both the foregoing general description and the following detailed description are exemplary and explanatory only and are not restrictive of the invention as claimed.

The accompanying drawings, which are incorporated in and constitute a part of this specification, illustrate one or more preferred embodiments of the invention and together with the description, serve to explain the principles of the invention.

### BRIEF DESCRIPTION OF THE DRAWINGS

FIG. 1 is a cross-sectional, side view of a cap for a vascular sheath according to the invention.

FIG. 2 is a top view of the cap of FIG. 1.

FIG. 3 is a partial cross-sectional, side view of a primary seal for a sheath according to the invention.

FIG. 4 is a cross-sectional, side view of a body of a vascular sheath according to the invention.

FIG. 5 is a side view of a secondary device that may be used with the invention.

FIG. 6 is a cross-sectional, side view of a vascular sheath according to the invention.

### DESCRIPTION OF PREFERRED EMBODIMENTS

Reference will now be made in detail to the preferred exemplary embodiments of the invention, examples of which are illustrated in the accompanying drawings.

As used herein, “distal” refers to being more distant to the operator (usually a surgeon) and closer to the interior of the patient's blood vessel, wherein “proximal” means closer to the operator and further from the interior of the patient's blood vessel.

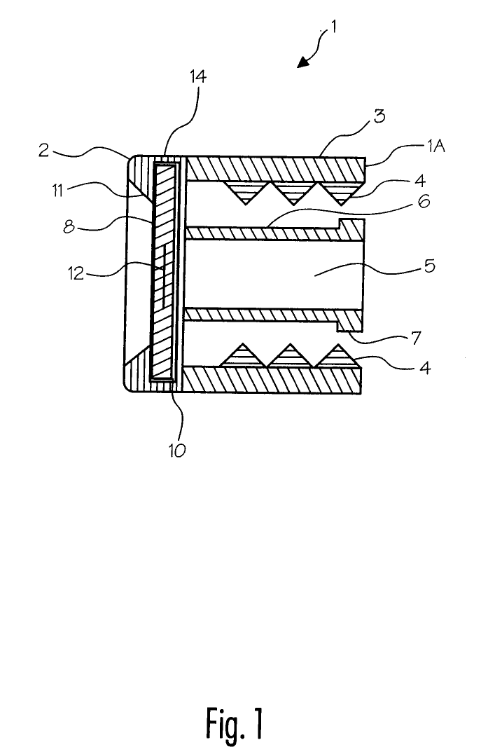

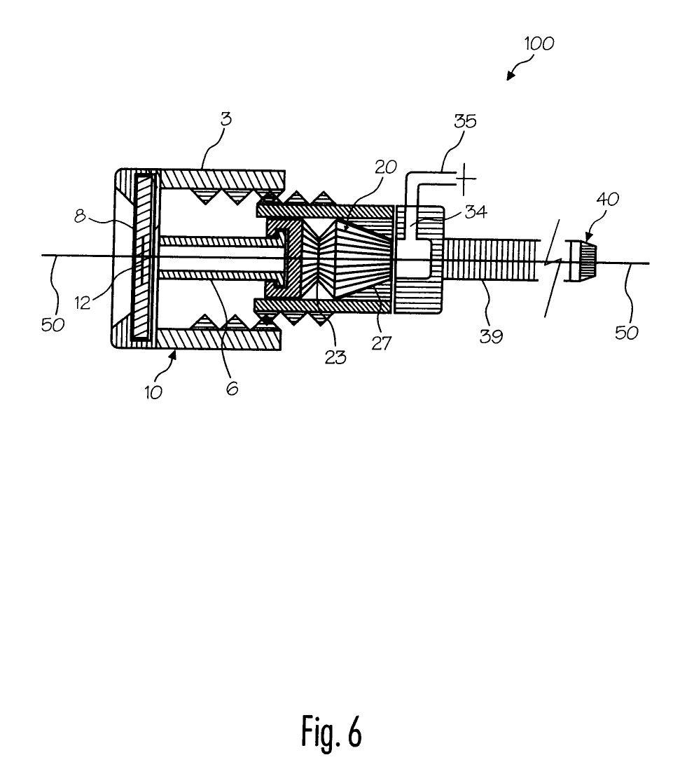

FIG. 1 is a cross-sectional, side view of a cap 1 showing a central post 6 and an enclosed thread 4 to threadibly engage a matching thread of body 30 of the vascular sheath 100 (see FIG. 6). The purpose of cap 1 is to seal sheath 100 and, in particular, to compress primary seal (or O-ring) 20, and any suitable structure may be used for this purpose. In this embodiment cap 1 is generally circular in shape.

Cap 20 is preferably comprised of injection molded plastic such as polyethylene, polypropylene or vinyl, but may be of any suitable material and manufactured using any suitable technique.

Central post 6 extends outward and has a flange or ridge 7 to which secondary seal 8 is preferably attached. Cap 1 has a distal end 1 A and a proximal end 2. Wall 3 of cap 1 and enclosed thread 4 are designed to engage a matching thread 37 of body 30, which is best seen in FIG. 6.

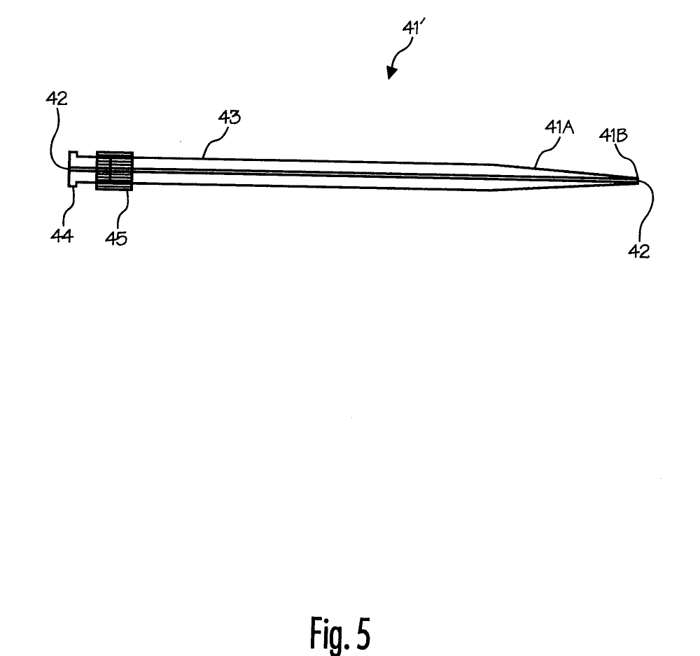

A central lumen 5 of post 6 extends from the base of cap 1 to the distal end of cap 1. Lumen 5 of post 6 is large enough to permit the passage of a secondary device, such as a large obdurator, an example of which is shown in FIG. 5. Bore 5 may have a diameter of, for example, 16 Fr, 18 Fr, 20 Fr, 22Fr, or 24 Fr. A secondary seal, as shown, is gasket 8, which has a slit or slits or other opening through which the secondary device may pass. Gasket 8 is preferably made of elastomeric silicone rubber although any suitable material may be used. In order to house gasket 8, proximal end 2 of cap 1 has a chamber 10 that receives gasket 8. Chamber 10 is preferably permanently closed once gasket 8 is positioned therein, but could be formed to open so that gasket 8 could be removed and changed. Angled edge 11 of cap 1 is optional and assists to facilitate centering of a secondary device (not shown in this Figure) passed through cap 1.

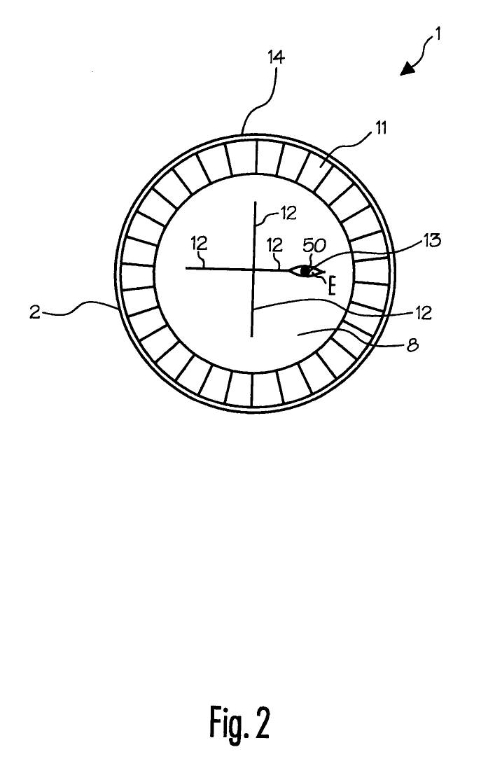

FIG. 2 is a top view of cap 1 and shows gasket 8 and the encircling edge or wall 11A that retains gasket 8. A circular lateral wall 14 on cap 2 retains gasket 8 laterally. As shown, a guide wire 50 passes through one or more slits 12 in gasket 8. The small eye-shaped defect E is, in this embodiment, the opening through which bleeding can occur. The slits 12 in gasket 8 are the openings through which a secondary device passes and these seal against the secondary device to help prevent bleeding. The distortion of these slits 12 (such as by a thin wire or angiocatheter) is how bleeding occurs with small-diameter secondary devices in relatively large bore vascular sheaths.

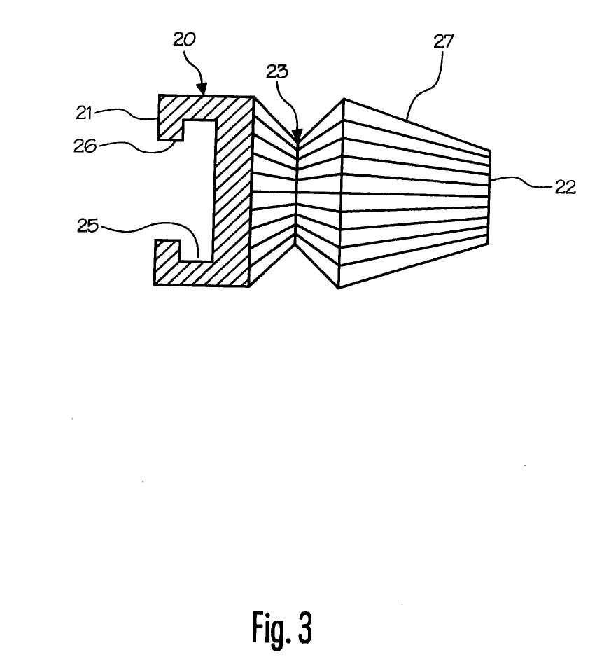

FIG. 3 is a view of the primary seal 20, which as shown is a modified O-ring that fits over flange 7 of cap 1. Seal 20 has a proximal end 21 and a distal end 22 with respect to the device and a body component 23. Seal 20 has a lumen (not shown) passing therethrough, the lumen sufficiently large to allow a secondary device to pass therethrough. Primary seal 20 is configured such that when mounted as part of sheath 100, and when compressed, at least part of the lumen constricts to substantially seal against the outer surface of a secondary device that may be present in the lumen. Seal 20 is preferably injection molded and made of elastomeric, silicone rubber, although any suitable material or method of manufacture may be utilized.

Proximal end 21 of seal 20 has a matching groove 25 and flange 26 to receive flange 7 of post 6 of cap 1, and post 6 compresses seal 20 when cap 1 is tightened on body 30 although any method or structure may be used to compress seal 20. Body part 23 of seal 20 has a conical distal end 27 that fits into a funnel chamber 31 of body 30 of vascular sheath 100. Seal 20 is sufficiently long and preferably has a crease and/or narrow diameter portion to allow seal 20 to collapse and further reduce the size of its lumen to accommodate small sized secondary devices such as guide wires or an angio-catheters.

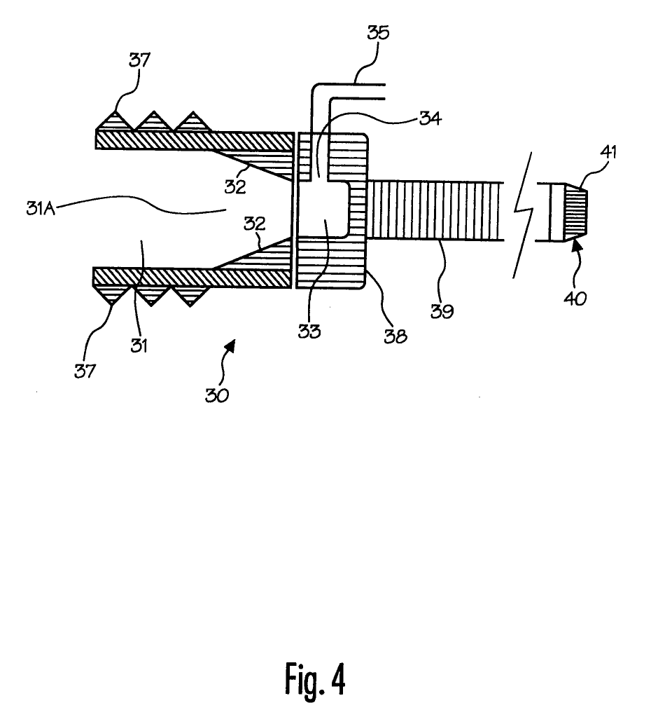

FIG. 4 is a cross-sectional view of a body 30 of the vascular sheath 100. Body 30 has a central chamber 31, which during use is preferably connected to a pressure line supporting a three-way stopcock for flushing, angiography or pressure monitoring while the sheath in place. Central chamber 31 receives seal 20, as shown in FIG. 6. The distal part 31A of chamber 31 is cone or funnel shaped, and has a wall 32 designed to receive the cone shaped distal end 27 (see FIGS. 3 and 6) of seal 20. Distal to chamber 31 is a second chamber 33 that is connected to an opening 34. Opening 34 feeds into a pressure line 35, which in turn is connected to a three-way stopcock (not shown) for access to the body 30 as required for such things as flushing, sampling, angiography via the vascular sheath, and taking hemodynamic measurements.

In this embodiment, external to central chamber 31 is external thread 37 that receives inner thread 4 of cap 1, so that cap 1 can be engaged and advanced or retracted on body 30 thus increasing or decreasing the compression on seal 20, and thus compressing or opening at least part of the lumen of seal 20, when desirable.

A rim 38, which is preferably circular, closes the chambers 31 and 33 from the air and connects to external sheath tube 39. Sheath tube 39 extends away from body 30 for an appropriate distance so that it can enter the blood vessel a distance required by the procedure being undertaken, for example, as far as the distal abdominal aorta or approximately as far as the orifices of one or both renal arteries and all positions in between from an entrance position at the common femoral artery.

The distal end 40 of sheath tube 39 preferably has a chamfered wall 41 so that it presents a low profile to produce little damage to the blood vessel wall when being inserted into the blood vessel. A small radio-opaque ring (not shown) preferably exists at end 40 so as to provide the operator with a x-ray visual understanding as to the precise position of the distal end of the sheath at all times during the procedure.

FIG. 5 is a side view of a secondary device, which in this case is an obdurator 41, that may be used with the invention. Obdurator 41 has a tapered distal end 41 A, which ends in a tip 41 B. A lumen 42 runs the entire length of obdurator 41 so that obdurator 41 can be passed over a guide wire. Body 43 of obdurator 41 is sized to match with an appropriately sized vascular sheath for a substantially hemostatic fit. In this example, the proximal end 41 of obdurator 44 is fitted with a Luer lock and gripping section 45 for easy grasping and removal or introduction.

FIG. 6 shows a preferred embodiment of an assembled vascular sheath 100 according to the invention. Sheath 100 has guide wire 50 passing therethrough, and, as shown, seal 20 is uncompressed. As cap 1 is screwed down on body 30, deformable (or compressible) body 23 of seal 20 will collapse to some degree and cone 27 will be pressed inward by pressure exerted by wall 32. At least part of the lumen of seal 20 will be forced to fully or substantially compress around the guide wire 50. Thus the small eye deformity (see FIG. 2) produced by wire 50 in gasket 8 will not leak blood because the blood is sealed by primary seal 20.

In summary, when a large diameter vascular sheath transmits a large secondary device, bleeding is usually not a major problem. However, to prevent bleeding when the large secondary device has been removed and the sheath only retains a small secondary device, such as a thin guide wire, the primary seal 20 should be compressed, thus fully or substantially compressing the lumen of seal 20 around the outside of the smaller secondary device to prevent bleeding. If a large secondary device needs to be reinserted the primary seal 20 is allowed to relax thereby opening its lumen.

Also, it is possible to increase the number of disk gaskets (in the preferred embodiment there is only one, gasket 8) and/or vary the style of slits from four to three or even one or possibly include a small single circular hole in one or more disk gaskets.

Another benefit that may be derived from the preferred embodiment of the invention is that it is simple to ship and store, and is fully assembled. The only step required is to flush the chamber access post via the side branch, which had a three-way stopcock at its end.

While this invention has been described in terms of its preferred embodiments and various modifications those skilled in the art can appreciate that other modifications can be made without departing form the spirit and scope of this invention. Other embodiments of the invention will be apparent to those skilled in the art from consideration of the specification and practice of the invention disclosed herein. It is intended that the specification and examples be considered as exemplary only, with a true scope and spirit of the invention being indicated by the ultimately-filed claims.

1. A vascular sheath having a manually compressible primary seal, the primary seal having a lumen therethrough for conforming to the outer dimensions of a secondary device passed through the lumen so as to prevent bleeding while the device is used in a blood vessel.

2. The vascular sheath of claim 1 that wherein the primary seal is elastomeric.

3. The vascular sheath of claim 1 that wherein the primary seal has a conical end.

4. The vascular sheath of claim 1 that further includes a secondary seal.

5. The vascular sheath of claim 4 wherein the secondary seal is manually movable.

6. The vascular sheath of claim 5 wherein the primary seal and secondary seal move in response to the same stimulus.

7. The vascular sheath of claim 4 wherein the secondary seal is elastomeric.

8. The vascular sheath of claim 4 wherein the secondary seal has one or more slits through which a secondary device may pass.

9. The vascular sheath of claim 8 wherein the secondary seal has two slits that form a cross.

10. The vascular sheath of claim 1 that includes a body and a cap mountable to the body.

11. The vascular sheath of claim 10 wherein the cap may be moved on the body once mounted thereon.

12. The vascular sheath of claim 11 wherein the cap includes a post.

13. The vascular sheath of claim 10 that further includes a secondary seal mounted to the cap.

14. The vascular sheath of claim 13 wherein the primary seal is mounted on the post.

15. The vascular sheath of claim 11 wherein the primary seal is compressed when the cap is moved in a first direction along the body and is relaxed when the cap is moved in a second direction along the body.

16. The vascular sheath of claim 10 wherein the primary seal is retained in a chamber in the body so that at least part of the lumen narrows when the primary seal is compressed.
